(19) United States
(12) Patent Application Publication
(10) Pub. No.: US 2011/0086387 A1
(43) Pub. Date: Apr. 14, 2011

(54) MUTANT NANOARCHAEUM EQUITANS A523R DNA POLYMERASE AND ITS USE

(76) Inventors: Suk Tae Kwon, Gyeonggi-do (KR); Jae Geun Song, Seoul (KR); Sung Suk Cho, Gangwon-do (KR)

(21) Appl. No.: 12/686,912

(22) Filed: Jan. 13, 2010

(30) Foreign Application Priority Data
Oct. 13, 2009 (KR) 10-2009-097261

(51) Int. Cl. C12P 21/06; C12P 19/34; C12N 9/12; C12N 1/21; C12N 15/63; C07H 21/04

(52) U.S. Cl. 435/69.1; 435/91.2; 435/194; 435/252.33; 435/320.1; 536/23.2

(57) ABSTRACT

Disclosed herein is a mutant Neq A523R DNA polymerase consisting of an amino acid sequence of SEQ ID NO: 2 wherein alanine at amino acid position 523 in a Nanoarchaeum equitans DNA polymerase (Neq DNA polymerase) consisting of an amino acid sequence of SEQ ID NO: 1 has been substituted with arginine by site-directed mutagenesis. Also disclosed are a gene consisting of a nucleotide sequence encoding the mutant Neq A523R DNA polymerase, a recombinant vector comprising the gene, and a transformant transformed with the vector. In addition, disclosed are a PCR kit comprising the mutant Neq A523R DNA polymerase having excellent performance compared to the wild-type Neq DNA polymerase, and a method for preparing the Neq A523R DNA polymerase.

### CROSS-REFERENCE TO RELATED APPLICATIONS

This application claims the benefit of Korean Patent Application No. 10-2009-097261, filed on, Oct. 13, 2009 in the Korean Intellectual Property Office, which is incorporated herein by reference in its entirety.

### BACKGROUND OF THE INVENTION

1. Field of the Invention

The present invention relates to a mutant Nanoarchaeum equitans DNA polymerase resulting from a mutation in a wild-type Nanoarchaeum equitans DNA polymerase, and the use thereof.

2. Background of the Related Art

Deoxyribonucleic acid polymerases (DNA polymerases; E.C. number 2.7.7.7) are enzymes that synthesize DNA in the 5′ to 3′ direction on template DNA. These enzymes play the most important role in DNA replication or repair in vivo.

DNA polymerases can be classified into at least six families (A, B, C, D, X and Y) on the basis of their amino acid sequences (Ohmori, H. et al., 2001, Mol Cell 8, 7-8). Most DNA polymerases belonging to the family B posses 3′ to 5′ exonuclease activity (so-called proofreading activity), and thus can achieve high-fidelity DNA replication. Among them, Archaeal family-B DNA polymerases contain in the N-terminal domain a specialized pocket that binds to deaminated bases such as uracil and hypoxanthine, causing the stalling of DNA replication (Fogg, M. J. et al., 2002, Nat Struct Biol, 9, 922-927; Gill, S. et al., 2007, J Mol Biol, 372, 855-863).

Nanoarchaeum equitans (Neq) is an extremely tiny (nano-sized), hyperthermophilic anaerobe isolated from a submarine hot-vent at the Kolbeinsey, north of Iceland (Huber, H. et al., 2002, Nature, 417: 63-67). This organism grows on the surface of a specific host, Ignicoccus sp. strain KIN4/I, under strictly anaerobic conditions.

A Neq DNA polymerase has a structure different from those of other family-B DNA polymerases. The Neq DNA polymerase is an Archaeal family-B DNA polymerase which has no a uracil-binding pocket in the N-terminal domain and can successfully utilize deaminated bases. The Neq DNA polymerase was first characterized by Choi, Jeong Jin, et al. (Choi, J. J., et al., 2008, Appl Environ Microbiol, 74: 6563-6569).

The Neq DNA polymerase is encoded by two genes, which are separated by 83,295 bp on the chromosome and individually contain a deduced split mini-intein sequence (Waters, E. et al., 2003, Proc Natl Acad Sci USA, 100: 12984-12988). In the prior art, a protein obtained by removing inteins through protein trans-splicing and linking only exteins by a peptide bond was designated “Neq C” (protein trans-spliced form of Neq DNA polymerase). Also, a DNA polymerase obtained in the form of a single polypeptide chain by combining an extein-encoding region of the Neq DNA polymerase large fragment gene, from which an intein-encoding region has been removed, with an extein-encoding region of the Neq DNA polymerase small fragment gene, from which an intein-encoding region has been removed, was designated “Neq P” (genetically protein splicing-processed form of Neq DNA polymerase). In addition, it was found that Neq C and Neq P, prepared by different methods, are enzymes exhibiting the same activity and biochemical properties (Choi, J. J. et al., 2006, J Mol Biol, 356:1093-106; Kwon, Suk-Tae, 2008, Korean Patent Registration No. 10-0793007). Furthermore, methods for preparing a Neq DNA polymerase and a Neq plus DNA polymerase (a mixture of Neq and Taq DNA polymerases) and the application of uracil-DNA glycosylase and dUTP to PCR were recently reported (Choi, J. J., et al., 2008, Appl Environ Microbiol, 74: 6563-6569).

Polymerase chain reaction (PCR) is a molecular biological technique and is very useful for detecting infection with virus or pathogenic bacteria. However, carry-over contamination may occur in a sample selection process, a nucleic acid separation process, a sample transfer process, a sample PCR process, a sample storage process, a sample collection process following electrophoresis, and the like. Such cross-over contamination can lead to false-positive results, when it is amplified together with a target sample, even if it is a very small amount. This results in a problem of lowering the accuracy of clinical diagnosis.

As a method for preventing cross-over contamination from occurring in PCR, a method of performing PCR in the presence of dUTP instead of dTTP was suggested (Longo, M. C. et al, 1990, Gene, 93:125-128). Also, methods of performing PCR comprising adding template DNA together with UDG for removing a very small amount of contaminant uracil-containing DNA in a sample, heating the mixture to inactivate the UDG, adding thereto dUTP instead of dTTP and performing PCR were reported (Rys, P. N., and D. H. Persing. 1993. J. Clin. Microbiol. 31:2356-2360). For this reason, PCR kit products which were treated with UDG in a PCR process separately or contain UDG in enzyme mixture have been marketed. Particularly in PCR which is carried out in the presence of dUTP instead of dTTP, a Taq DNA polymerase is mainly used.

Recently, the PCR efficiency of a Neq DNA polymerase and its application to PCR were reported. However, the Neq DNA polymerase has a problem in that it has low amplification efficiency compared to the Taq DNA polymerase (Choi, J. J., et al., 2008, Appl. Environ. Microbiol., 74: 6563-6569).

### SUMMARY OF THE INVENTION

The present invention is to provide a mutant Neq A523R DNA polymerase consisting of an amino acid sequence of SEQ ID NO: 2 wherein alanine at amino acid position 523 in a Nanoarchaeum equitans DNA polymerase (hereinafter also referred to as Neq DNA polymerase) consisting of an amino acid sequence of SEQ ID NO: 1 has been substituted with arginine by site-directed mutagenesis.

The present invention is also to provide a gene consisting of a nucleotide sequence encoding said mutant Neq A523R DNA polymerase, a recombinant vector comprising the gene, and a transformant transformed with the recombinant vector.

The present invention is also to provide a PCR kit comprising said mutant Neq A523R DNA polymerase.

The present invention is also to provide a primer set consisting of an A523R-F primer of SEQ ID NO: 4 and an A523R-R primer of SEQ ID NO: 5, which are used in the preparation of said mutant Neq A523R DNA polymerase.

The present invention is also to provide a method for preparing said mutant Neq A523R DNA polymerase.

The present invention is also to provide a method of performing polymerase chain reaction (PCR) using said mutant Neq A523R DNA polymerase.

### BRIEF DESCRIPTION OF THE DRAWINGS

FIG. 1 shows a comparison of the processivity of a Neq A523R DNA polymerase with a wild-type Neq polymerase. FIG. 1A shows electropherograms of the two DNA polymerases, wherein each peak indicates an amplified fragment of each polymerase. FIG. 1B is a graphic representation showing log (Aₙₜ/A_{T}) vs. extended length for each peak of FIG. 1 (o: wild-type Neq, and •: Neq A523R DNA).

FIG. 2 shows a comparison of the extension rate of the Neq A523R DNA polymerase with the wild-type Neq polymerase. The extension rates of the two polymerases were calculated as the length of DNA amplified per hour.

FIG. 3 shows a comparison of the PCR efficiency of the Neq A523R DNA polymerase with the wild-type Neq and Neq A523R DNA polymerase. The size of amplified DNA is shown at the bottom of the figure. FIG. 3A shows the results of amplification obtained with an extension time of 90 seconds, and FIG. 3B shows the results of amplification obtained with an extension time of 3 minutes. FIG. 3C shows a comparison of the PCR efficiency of the Neq A523R DNA polymerase with the Taq DNA polymerase in the presence of dUTP.

FIGS. 4 to 9 show the results obtained by performing real-time PCR targeting a 200-bp fragment of human β-actin DNA. The Neq A523R DNA polymerase is depicted as a solid line, and the Taq DNA polymerase is depicted as a dotted line. FIGS. 4, 5 and 6 show the results of amplification performed using dNTP. Specifically, FIG. 4 is an amplification curve, FIG. 5 is a melting curve, and FIG. 6 shows the results of amplification of amplified products. FIGS. 7, 8 and 9 shows the results of amplification performed in the presence of dUTP. Specifically, FIG. 7 is an amplification curve, FIG. 8 is a melting curve, and 9 shows the results of electrophoresis of amplified products.

### DETAILED DESCRIPTION OF THE PREFERRED EMBODIMENT

The present inventors predicted the three-dimensional structure of Neq DNA polymerase by comparing the Neq DNA polymerase with family-B DNA polymerases which have significantly high sequence homology with the Neq DNA polymerase and whose three-dimensional structures have already been elucidated (Hashimoto, H., et al., 2001, J. Mol. Biol. 306: 469-477; Hopfner, K. P., et al., 1999, Proc. Natl. Acad. Sci. USA, 96: 3600-3605; Rodriguez A. C., et al., 2000, J. Mol. Biol. 299: 447-462).

The present invention provides a Neq A523R DNA polymerase having improved performance as a result of substituting alanine at position 523 (A523) of the fingers subdomain of the Neq DNA polymerase with another residue by site-directed mutagenesis, and a preparation method thereof.

The Neq A523R DNA polymerase prepared according to the present invention has processivity and extension rate which are about three-times higher than those of the existing Neq DNA polymerase, and it can more efficiently perform PCR. Based on this fact, the present inventors have found that the mutant Neq A523R DNA polymerase can be used more efficiently than the Taq DNA polymerase in real-time PCR and general PCR, which are performed using dUTP, thereby completing the present invention.

An object of the present invention is to provide a mutant Neq A523R DNA polymerase consisting of an amino acid sequence of SEQ ID NO: 2 wherein alanine at amino acid position 523 of an Neq DNA polymerase of SEQ ID NO: 1 has been substituted with arginine by site-directed mutagenesis.

Another object of the present invention is to provide a gene consisting of a nucleotide sequence encoding said mutant Neq A523R DNA polymerase, a recombinant vector comprising the gene, and a transformant transformed with the recombinant vector.

Still another object of the present invention is to provide a PCR kit comprising said mutant Neq A523R DNA polymerase.

Still another object of the present invention is to provide a primer set consisting of an A523R-F primer of SEQ ID NO: 4 and an A523R-R primer of SEQ ID NO: 5, which are used in the preparation of the Neq A523R DNA polymerase.

Still another object of the present invention is to provide a method for preparing said mutant Neq A523R DNA polymerase.

Yet another object of the present invention is to provide a method of performing polymerase chain reaction (PCR) using said Neq A523R DNA polymerase.

The X-ray three-dimensional structures of family-B DNA polymerases have already been elucidated. Because the amino acid sequences of the family-B DNA polymerases and the Neq DNA polymerase have very high homology, their structures were predicted to be very similar. From the Neq DNA polymerase structure based on this prediction, a mutant protein was produced by mutating alanine at position 523 (hereinafter referred to as “A523”) in the fingers subdomain of the Neq DNA polymerase by site-direction mutagenesis through overlapping PCR, and a recombinant vector was constructed by cloning the prepared mutant gene into a pET-22b(+) vector.

The present invention comprises a gene consisting of a nucleotide sequence encoding the mutant Neq A523R DNA polymerase. The gene is preferably a gene having a base sequence of SEQ ID NO: 3.

In another aspect, the present invention comprises a recombinant vector comprising a gene encoding the mutant Neq A523R DNA polymerase, and a transformant transformed with the recombinant vector.

As used herein, the term “vector” refers to a means by which DNA is introduced into a host cell for protein expression. Examples of the vector include all ordinary vectors such as plasmid vectors, cosmid vectors, bacteriophage vectors, and viral vectors. The vector is preferably a plasmid vector.

A suitable expression vector may comprise expression regulatory elements, such as a promoter, an initiation codon, a stop codon, a polyadenylation signal and an enhancer. It is necessary that the initiation and stop codons must be functional in the individual to whom the gene construct is administered. Also, the initiation and stop codons must be in frame with the coding sequence. The promoter may be constitutive or inducible, and a replicable expression vector may include an origin of replication. In addition, the expression vector may include selectable markers that confer selectable phenotypes, such as selectable phenotypes, such as drug resistance, auxotropy, resistance to cytotoxic agents, or surface protein expression.

Specifically, the present invention comprises a pENPA523R recombinant vector expressing the mutant Neq A523R DNA polymerase having an amino acid sequence of SEQ ID NO: 2. In addition, the present invention also comprises a transformant transformed with the vector.

A microorganism usable as a host cell which can be transformed with the vector is not specifically limited. Examples of the microorganism include various microorganisms such as Escherichia coli, Rhodococcus, Pseudomonas, Streptomyces, Staphylococcus, Sulfolobus, Thermoplasma, and Thermoproteus. E. coli is preferably used, and examples thereof include, but are not limited to, E. coli XL1-blue, E. coli BL21(DE3), E. coli JM109, E. coli DH series, E. coli TOP10 and E. coli HB101. Specifically, the present invention comprises Escherichia coli BL21-CodonPlus(DE3)-RIL/pENPA523R transformed with pENPA523R. The above Escherichia coli BL21-CodonPlus(DE3)-RIL/pENPA523R was deposited at the Korean Culture Center of Microorganisms (#361-221, Hongje-1-dong, Seodaemun-gu, Seoul, Korea) on Aug. 19, 2009.

Methods for transforming the vector into host cells include any method by which nucleic acids can be introduced into cells, and the transformation of the vector into host cells can be performed using suitable standard techniques selected according to host cells. These methods include, but are not limited to, electroporation, protoplast fusion, calcium phosphate (CaPO₄) precipitation, and calcium chloride (CaCl₂) precipitation.

The Neq A523R DNA polymerase of the present invention can be used as one component of a PCR kit. The PCR kit of the present invention comprises, in addition to the Neq A523R DNA polymerase, one or more other components, solutions or devices. For example, the kit of the present invention may comprise at least one component selected from among a vessel containing detection primers, an amplification tube or container, a reaction buffered solution, dNTPs, RNase and sterile water.

The kit containing the Neq A523R polymerase of the present invention can be more useful than the Taq DNA polymerase in various fields, including genetic and molecular biological experiments, clinical diagnosis and medicolegal research.

Furthermore, the present invention comprises a method for preparing the mutant Neq A523R DNA polymerase.

The mutant Neq A523R DNA polymerase can be prepared through a process comprising the steps of: (i) preparing a recombinant vector expressing the mutant Neq A523R DNA polymerase; (ii) transforming a host cell with the recombinant vector to obtain a transformant; (iii) culturing the transformant; and (iv) harvesting a DNA polymerase from the transformant.

The kind of vector used in step (i), a method for transformation with the vector in step (ii) and the kind of microorganism used in step (ii) are as described above.

The culture process of step (iii) is carried out according to a conventional method under conditions allowing expression of the cloned gene. This culture process can be easily adjusted depending on the kind of microorganism selected. The medium that is used in the culture process will usually contain all nutrients necessary for the growth and survival of the cells. The medium may contain various carbon sources, nitrogen sources, trace elements and the like. The culture temperature and time of the transformant can be adjusted depending on the culture conditions.

Also, an inducer such as isopropyl-β-D-thiogalactopyranoside (hereinafter referred to as “IPTG”) may be used to induce protein expression. The kind of inducer used can be determined depending on the vector system, and conditions such as the injection time and amount of the inducer can be suitably adjusted.

In step (iv), the protein can be purified using a conventional method. For example, the cells are harvested by centrifugation and disrupted using, for example, a sonicator, and the disrupted cells are centrifuged to remove cell debris, thus obtaining the supernatant. The solution containing the protein obtained from the host cell is purified using conventional techniques, including salting out, solvent precipitation, dialysis and chromatography (e.g., gel filtration chromatography, ion exchange chromatography or affinity chromatography), which are used alone or in combination, thereby obtaining the Neq A523R DNA polymerase protein of the present invention.

In Examples of the present invention, each of heat-treated samples was centrifuged, and only the supernatant was collected, dialyzed, and then purified using an anion-exchange column (HiTrap Q) and a cation-exchange column (HiTrap SP).

The inventive mutant Neq A523R DNA polymerase prepared as described above has the following characteristics: processivity and extension rate which are about three times higher than those of the wild type Neq DNA polymerase; and improved PCR efficiency. In other words, the present invention can provide a mutant Neq A523R DNA polymerase having improved performance compared to the wild-type polymerase.

Furthermore, the present invention comprises a method of performing PCR using the Neq A523R DNA polymerase.

The Neq A523R DNA polymerase of the present invention shows excellent PCR efficiency compared to the prior Neq DNA polymerase. Also, the Neq A523R DNA polymerase can perform PCR in the presence of dUTP, like the case of the Taq DNA polymerase. Particularly, it shows excellent amplification efficiency compared to the Taq DNA polymerase in the presence of dUTP and can perform PCR in a shorter time.

When the Neq A523R DNA polymerase of the present invention performs PCR using human genomic DNA as a template in the presence of dUTP, it shows excellent specificity compared to the Taq DNA polymerase. In other words, it can specifically amplify only the desired target fragment and has excellent amplification efficiency. Particularly, the Neq A523R DNA polymerase which is provided according to the present invention as described above has excellent polymerization activity compared to existing polymerases (e.g., Taq DNA polymerase) in the presence of dUTP, and thus will be very suitable for real-time PCR which is performed using UDG and dUTP in order to, for example, diagnose diseases.

The Neq A523R DNA polymerase of the present invention has processivity and extension rate, which are about three times higher than those of the existing Neq DNA polymerase. Particularly, in the presence of deoxy UTP (dUTP), the Neq A523R DNA polymerase shows excellent amplification efficiency and specificity compared to the Taq DNA polymerase which is most frequently used in PCR. The mutant polymerase of the present invention can be used more efficiently than existing polymerases in the technique for preventing carry-over contamination using deoxy UTP (dUTP) together with uracil-DNA glycosylase (hereinafter referred to as “UDG”).

Hereinafter, the present invention will be described in further detail with reference to the following examples. It is to be understood, however, that these examples are for illustrative purposes only and are not to be construed to limit the scope of the present invention.

### EXAMPLES

### Example 1

### Construction of Recombinant Plasmid

A point mutation in the wild-type Neq DNA polymerase gene was induced through an overlapping PCR method using the primers (A523R-F and A523R-R oligonucleotides) shown in Table 1 below (see Ho, S. N. et al., 1989, Gene, 77:51-59). As a result, the mutant polymerase gene had a sequence wherein alanine (Ala) at amino acid position 523 has been substituted with arginine (Arg).

The mutant gene was amplified using a NeqFP primer (SEQ ID NO. 16: 5′-ATTATAGCATATGTTACACCAACTCCCCACG-3′ wherein the underlined portion represents the Nde I restriction enzyme recognition site) and a NeqRP primer (SEQ ID NO. 17: 5′-AAAAAACTAACAGATTTCTTTAAATGAGTCGACATTAT-3′ wherein the underlined portion represents the Sal I restriction enzyme recognition site), and the amplified gene was cloned into a pET-22b(+) plasmid (Novagen, USA) containing a T7 lac promoter. The plasmid was transformed into E. coli DH5α (Takara Bio), thus preparing a recombinant vector containing the mutant Neq DNA polymerase having an alanine-to-arginine substitution at amino acid position 523. Also, it was confirmed by DNA sequencing that alanine at position 523 of the Neq DNA polymerase gene cloned into the recombinant vector was accurately substituted with arginine.

The E. coli strain BL21-CodonPlus(DE3)-RIL (Stratagene, USA), which harbors the T7 RNA polymerase under the control of a chromosomal lacUV5 gene, was selected as a host for gene expression, and the above plasmid vector was transformed into the selected E. coli host by electroporation. The recombinant vector comprising the gene encoding the Neq A523R DNA polymerase was named “pENPA523R”. The expression vector containing the Neq A523R DNA polymerase gene, and E. coli containing the expression vector were named “Escherichia coli BL21-CodonPlus(DE3)-RIL/pENPA523R”. The Escherichia coli BL21-CodonPlus(DE3)-RIL/pENPA523R was deposited at the Korean Culture Center of Microorganisms on Aug. 19, 2009 under accession number KCCM11031P.

TABLE 1

| | | |
|---|---|---|
| Oligonucleotide | | SEQ |
| Name | Oligonucleotide Sequence* | ID NO. |

Primers used in overlapping PCR

| | | | |
|---|---|---|---|
| A523R | F | TGCTAAGCAAAGAGTATTGAAAATAATAATTAATGCAAC | 4 |
| | R | TTTTCAATACTCTTTGCTTAGCATTTATAATTTTATATTC | 5 |

Primers used in PCR Efficiency Test

| | | | |
|---|---|---|---|
| λ-1 | F | AATAACGTCGGCAACTTTGG | 6 |
| (0.5 kb) | R | GTTACGCCACCAGTCATCCT | 7 |
| λ-2 | F | CAAAGGCGGTTAAGGTGGTA | 8 |
| (1 kb) | R | GGCTGTACCGGACAATGAGT | 9 |
| λ-3 | F | AGAAGTTCAGGAAGCGGTGA | 10 |
| (2 kb) | R | ACGGAAAAAGAGACGCAGAA | 11 |
| λ-4 | F | CCGGTAATGGTGAGTTTGCT | 12 |
| (4 kb) | R | GTACTGGTGCCTTTGCCATT | 13 |
| λ-5 | F | TTTACAGCGTGATGGAGCAG | 14 |
| (6 kb) | R | GACACGGTGGCTTTTGTTTT | 15 |

All the primers have the 5′ to 3′ direction.
Primer F means a forward primer, and primer R means a reverse primer.

### Example 2

### Expression and Purification of Mutant Enzyme

An E. coli BL21-CodonPlus(DE3)-RIL strain having the recombinant plasmid constructed in Example 1 was inoculated into 500 Ml of LB medium supplemented with 100 μg/Ml of ampicillin was cultured at 37 until the absorbance at a wavelength of 600 nm reached about 0.6. Then, in order to induce expression of the cloned gene, IPTG was added to the cultured cells to a final concentration of 0.2 mM, and the cells were cultured again at 37 for 6 hours. Next, the cells were harvested by centrifugation, and the harvested cells were suspended and sonicated in buffer A (20 mM sodium phosphate (pH 6.0)/50 mM KCl) containing 1 mM phenylmethylsulfonyl fluoride (hereinafter referred to as “PMSF”) as a protease inhibitor. Then, the sonicated extract was separated by centrifugation into the supernatant and the pellet. The supernatant was heat-treated at 80 for 30 minutes and separated by centrifugation into the supernatant and the pellet. The supernatant was dialyzed in buffer A (20 mM sodium phosphate (pH 6.0)/50 mM KCl), and then passed through an anion-exchange column (HiTrap Q; GE Healthcare) to remove most proteins other than the target protein. The buffer containing the purified proteins was bound to a cation-exchange column (HiTrap SP; GE Healthcare), and then the proteins were purified using buffer B (20 mM sodium carbonate (pH 10.5), 50 mM KCl) while increasing the pH. The fractions containing the purified proteins were collected and dialyzed in buffer A, and then the proteins were quantified by the Lowry method (Lowry, O. H. et al., 1951. J Biol Chem, 193, 265-275).

### Example 3

### Comparison of Processivity of Neq DNA Polymerase and Neq A523R DNA Polymerase

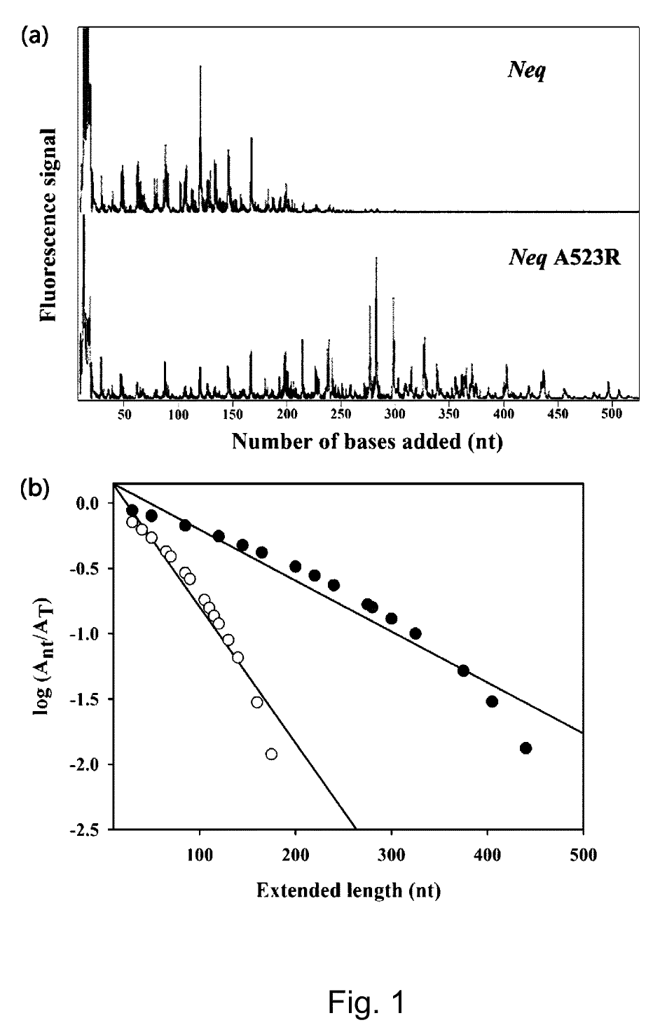

To elucidate the cause of the improved PCR efficiency of the Neq A523R DNA polymerase, a processivity assay was carried out. A reaction solution containing 400 fmol of an M13 primer (SEQ ID NO. 18: 5′-CCGTTGCTACCCTCGTTCCGATGC-3′) having Hex at the 5′ end, 200 fmol of M13 single-stranded DNA (NEB), Neq DNA polymerase buffer and 0.2 mM dNTPs was heated at 95 for 1 minute and subjected to primer annealing at for 1 minute. Then, DNA was added to the reaction solution at a concentration of 4 pmole. The reaction solution was subjected to an enzymatic reaction at 75 for seconds, and then the synthesized DNA fragments were analyzed on an ABI3100 automated sequencer (Kim, Y. J. et al. 2007. J Microbiol Biotechnol, 17: 1090-1097). The processivities of the Neq DNA polymerase and the Neq A523R DNA polymerase were displayed as electropherograms in FIG. 1A. The peaks shown in the electropherograms mean one amplified DNA fragment, and the highest peak around the average extended length was defined as the processivity of each DNA polymerase. As a result, the wild-type Neq DNA polymerase showed 120 nt (nucleotides), and the Neq A523R DNA polymerase showed a processivity of 280 nt. The processivity of a polymerase can also be defined as the probability of the polymerase not terminating at a specific position of the template (Von Hippel, P. H. et al. 1994. Ann N Y Acad Sci, 726: 118-131). The peaks shown in FIG. 1A were expressed as a logarithmic function in FIG. 1B. The probability of the processivity of each polymerase was calculated from the slope shown in the graph of FIG. 1B, and as a result, it was 0.976 for the Neq DNA polymerase and 0.991 for the Neq A523R DNA polymerase. The average extended length can be calculated as 1/(1−P) and was 42.17 nt for the Neq DNA polymerase and 111.61 nt for the Neq A523R DNA polymerase. All values concerning processivity, calculated from the experimental results, are summarized in Table 2 below. These results indicate that the processivity of the Neq A523R DNA polymerase was increased about three times compared to that of the wild-type Neq DNA polymerase. It was previously reported that the processivity of a DNA polymerase greatly influences the performance of the polymerase, and particularly has a great effect on the increase in PCR efficiency (see Wang Y., et al., 2004, Nucleic Acids Res, 32, 1197-1207).

TABLE 2

| DNA | Processivity | Fine Processivity | Average Extended |
|---|---|---|---|
| polymerase | (nt) | (P) | Length (nt) |
| Neq (wild- | 120 | 0.976 | 42.17 |
| type) | | | |
| Neq A523R | 280 | 0.991 | 111.61 |

### Example 4

### Comparison of Extension Rate of Neq DNA Polymerase and Neq A523R DNA Polymerase

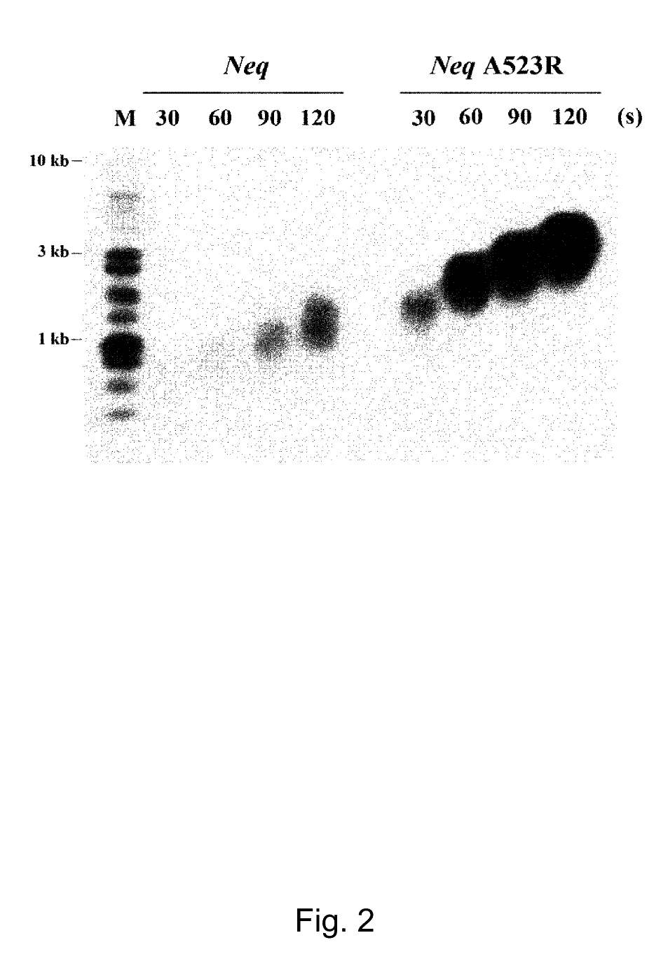

The extension rates of the DNA polymerases were calculated from the length of DNA synthesized in a fixed time (Takagi, M. et al. 1997. Appl Environ Microbiol, 63: 4504-4510). 50 μl of a reaction solution contained 1.25 ng of M13 single-stranded DNA (NEB), 10 pmol of M13 primer (SEQ ID NO. 19: 5′-GCATCGGAACGAGGGTAGCAACGG-3′), 250 mM of each of dATP, dTTP and dGTP, 25 mM of dCTP, 10 mCi of [α−³²P] dCTP (800 Ci/mmol, PerkinElmer), 0.4 mg of each DNA polymerase, and a buffer for each Neq DNA polymerase. The reaction solution was incubated at 75 for each of 30, 60, 90 and 120 seconds. Next, 10 μl of the reaction solution was taken and an equal amount of stop solution (containing 60 mM EDTA and 60 mM NaOH) was added to stop the reaction. A 10-μl aliquot of each sample was analyzed by agarose gel electrophoresis, and the analysis results were visualized with X-ray films. As a result, the Neq DNA polymerase showed an extension rate of about 12 nt (nucleotides)/second, and the Neq A523R DNA polymerase showed an extension rate of 35 nt/s (see FIG. 2).

### Example 5

### Analysis of PCR Efficiencies of DNA Polymerases

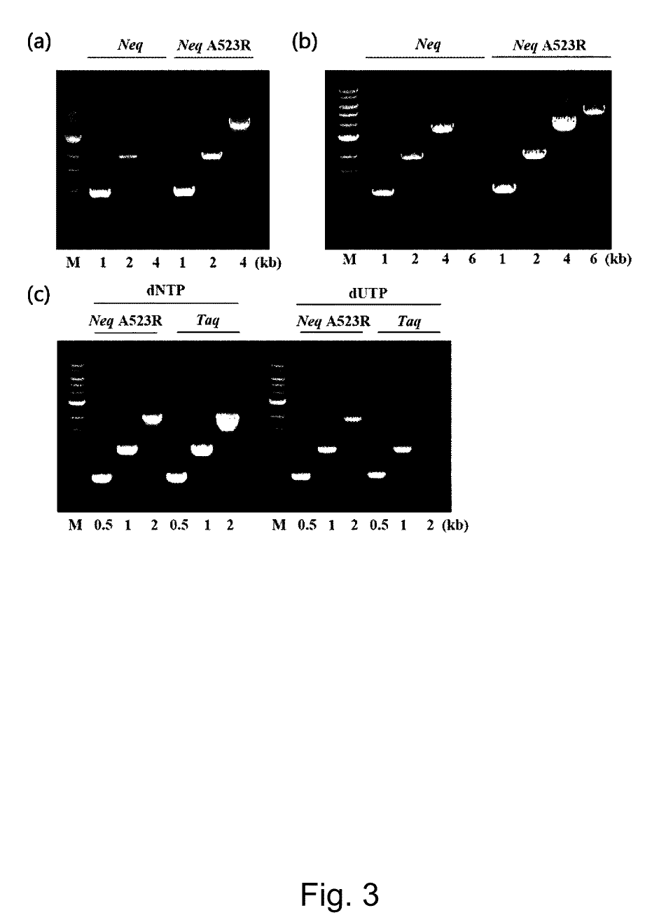

In order to compare the PCR efficiency of Neq and Neq A523R DNA polymerases, primers for amplifying DNA fragments having various lengths of 1 kb, 2 kb, 4 kb and 6 kb were added to perform PCR amplification (forward and reverse λ-1, λ-2, λ-3, λ-4 and λ-5 primers; see Table 1). 1 pmole of each DNA polymerase, 5 pmole of each primer, 250 μM of dNTP and 30 ng of lambda DNA were added to 20 μl of each PCR reaction solution to perform PCR amplification. The PCR amplification was performed under the following conditions: DNA denaturation at 94 for 3 minutes; and then 30 cycles each consisting of DNA denaturation at 94 for 20 seconds and DNA extension at 72 for 90 seconds and 3 minutes; followed by final extension at 72 for 3 minutes. The Neq DNA polymerase could amplify 2-kb lambda DNA for an extension time of 90 seconds, whereas the Neq A523R DNA polymerase could amplify 4-kb lambda DNA for the same time (FIG. 3A). Also, for an extension time of 3 minutes, the Neq DNA polymerase could amplify a 4-kb DNA fragment, and the Neq A523R DNA polymerase could amplify a 6-kb DNA fragment. In addition, the Neq A523R DNA polymerase could perform amplification with a higher yield. As a result, the Neq A523R DNA polymer showed higher amplification yield and efficiency than those of the wild-type Neq DNA polymerase (see FIG. 3B).

In order to examine the amplification efficiencies of the Neq A523R DNA polymerase and the Taq DNA polymerase in the presence of dUTP, 250 μM of dNTP (replaced dTTP with dUTP) and each of primers for amplifying DNA fragments having various lengths of 0.5 kb, 1 kb and 2 kb were added to perform PCR amplification (other reaction solution conditions were the same as the above-described experimental conditions). The Taq DNA polymerase was used in PCR amplification according to the optimal protocol provided by the manufacturer (TaKaRa Bio). The PCR amplification was performed under the following conditions: DNA denaturation at for 3 minutes; and then 25 cycles each consisting of DNA denaturation 94 for 20 seconds, primer annealing at 56 for 20 seconds and DNA extension at 72 for 30 seconds; followed by final extension at 72 for 3 minutes. In the experiment conducted using dNTP as a substrate, the Taq DNA polymerase showed slightly excellent PCR efficiency compared to the Neq A523R DNA polymerase, but in the experiment conducted using dUTP as a substrate, the Neq A523R DNA polymerase could amplify a longer DNA fragment with a higher yield for the same amplification time (see FIG. 3C).

### Example 6

### Real-Time PCR

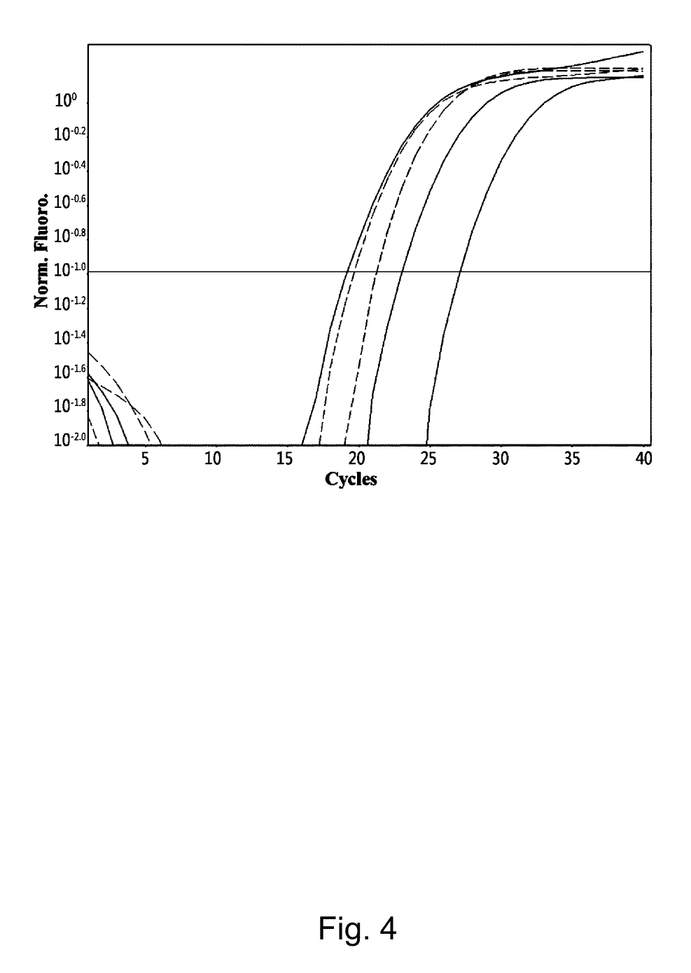

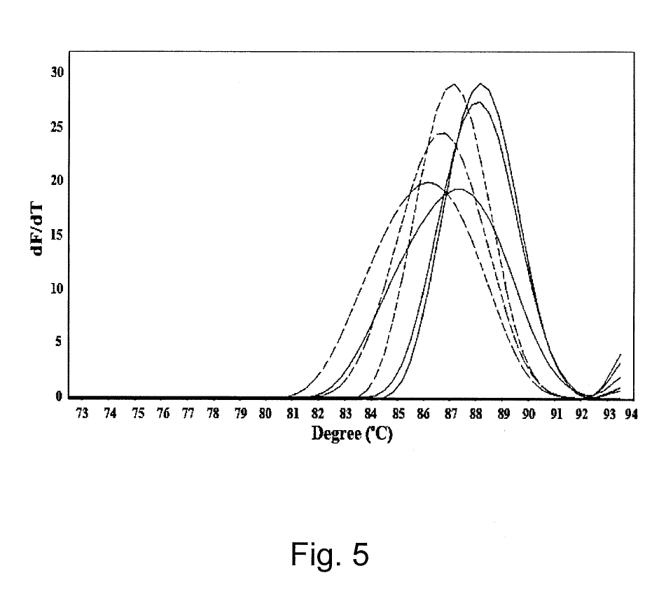

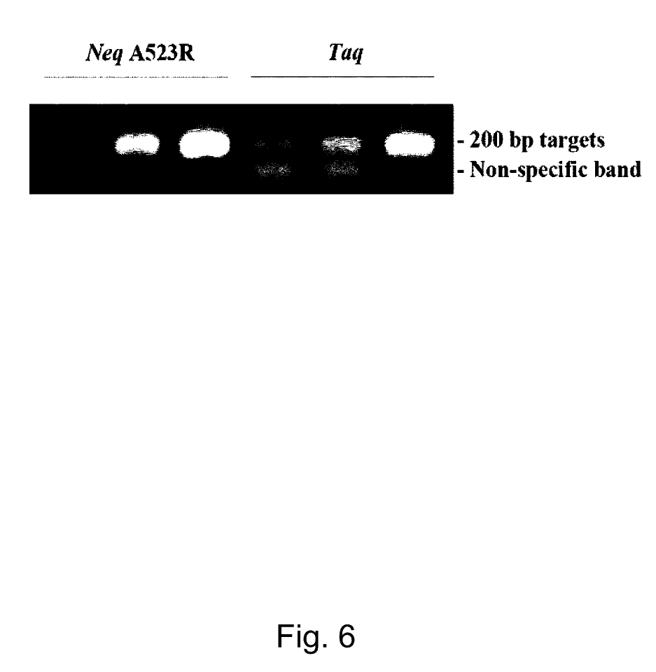

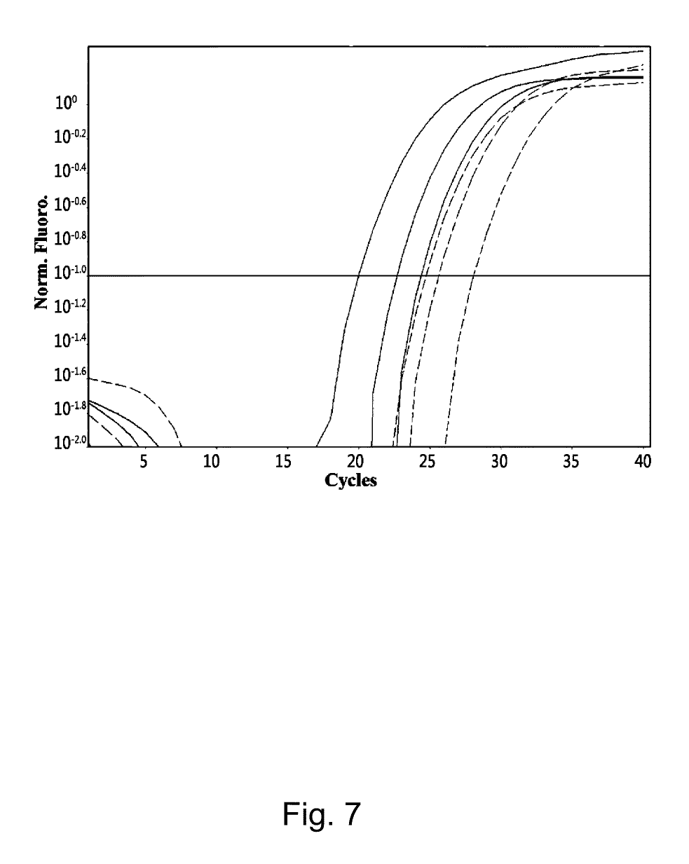

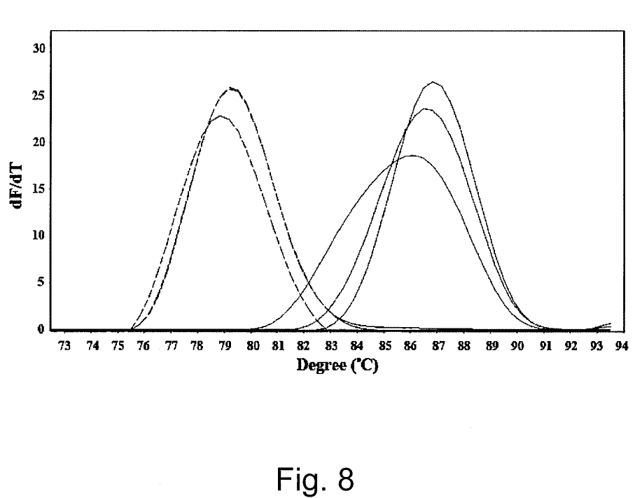

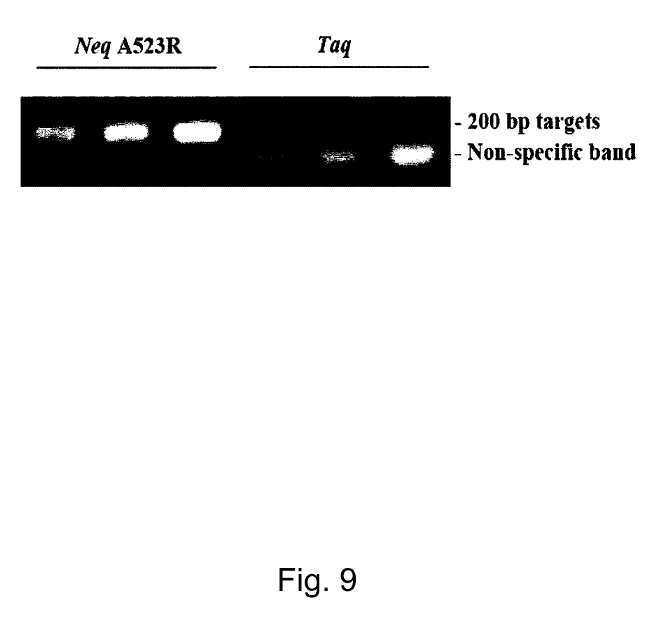

Quantitative real-time PCR was performed by measuring the amount of SYBR green inserted into DNA. A reaction solution contained 5 pmol of each of forward primer (SEQ ID NO. 20: 5′-AGAGATGGCCACGGCTGCTT-3′) and reverse primer (SEQ ID NO. 21: 5′-ATGATGGAGTTGAAGGTAGT-3′) targeting a 200-bp fragment of human genomic β-actin DNA, each of 0.1 ng, 1 ng and 10 ng of human genomic DNA, 250 μM of dNTPs (dATP, dCTP, dGTP, dTTP or dUTP), and 1×SYBR green. Each of 1 pmole of the Neq A523R polymerase and 1 U of the Taq DNA polymerase was added to the reaction solution to perform PCR amplification. The PCR amplification was performed using PCR Rotor-gene RG-3000 (Corbett Research) under the following conditions: initial denaturation at 95 for 3 minutes; and then 40 cycles each consisting of denaturation at 94 for 20 seconds, annealing at 60 for 20 seconds and extension at 72 for 20 seconds; followed by final extension at 72 for 2 minutes. The produced amplification products were finally electrophoresed to determine their lengths. In the experiment conducted using dNTP, the number of threshold cycles (Ct) was lower in the Taq DNA polymerase (20.69) than in the Neq A523R polymerase (23.08) (see FIG. 4). In the melting curve analysis, it could be seen that the Taq DNA polymerase resulted in increased non-specific amplification compared to the Neq A523R polymerase (see FIG. 5). This is more evident from the results of electrophoretic analysis of the amplified products (see FIG. 6). In the same manner as described above, real-time PCR was performed using dUTP instead of dTTP. As a result, the Neq A523R polymerase showed a Ct value of 22.33, whereas the Taq DNA polymerase showed a Ct value of 26.13 (see FIG. 7). This suggests that the amplification efficiency of the Taq DNA polymerase in the presence of dUTP is lower than that of the Neq A523R polymerase. In addition, through melting curve analysis (FIG. 8) and agarose gel electrophoretic analysis of the amplified products (FIG. 9), it could be seen that the Neq A523R polymerase could more specifically perform PCR compared to the Taq DNA polymerase.

What is claimed is:

1. A mutant Neq A523R DNA polymerase consisting of an amino acid sequence of SEQ ID NO: 2 wherein alanine at amino acid position 523 in a Neq DNA polymerase consisting of an amino acid sequence of SEQ ID NO: 1 has been substituted with arginine by site-directed mutagenesis.

2. A gene consisting of a nucleotide sequence encoding the mutant Neq A523R DNA polymerase of claim 1.

3. The gene of claim 2, wherein the nucleotide is a gene having a base sequence of SEQ ID NO: 3.

4. A recombinant vector comprising a gene encoding the mutant Neq A523R DNA polymerase.

5. The recombinant vector of claim 4, wherein the recombinant vector is a pENPA523R recombinant vector.

6. A transformant transformed with the pENPA523R recombinant vector of claim 5.

7. The transformant of claim 6, wherein the transformant transformed with the pENPA523R recombinant vector is Escherichia coli BL21-CodonPlus(DE3)-RIL/pENPA523R (deposited under accession number KCCM11031P).

8. A PCR kit comprising the mutant Neq A523R DNA polymerase.

9. A primer set consisting of an A523R-F primer of SEQ ID NO: 4 and an A523R-R primer of SEQ ID NO: 5, which are used to substitute alanine with arginine in the preparation of said mutant Neq A523R DNA polymerase.

10. A method for preparing a mutant Neq A523R DNA polymerase, the method comprising the steps of:
(i) preparing a recombinant vector expressing the mutant Neq A523R DNA polymerase of claim 1;
(ii) transforming a host cell with the recombinant vector to obtain a transformant;
(iii) culturing the transformant; and
(iv) harvesting a DNA polymerase from the transformant.

11. Method of claim 10, wherein the step of preparing a recombinant vector expressing the mutant Neq A523R DNA polymerase of claim 1 comprises using a primer set consisting of an A523R-F primer of SEQ ID NO: 4 and an A523R-R primer of SEQ ID NO: 5 in order to substitute alanine with arginine.

12. A method of performing polymerase chain reaction (PCR) using the Neq A523R DNA polymerase of claim 1.

13. The method of claim 12, wherein the polymerase chain reaction (PCR) is performed in the presence of dUTP (2′-deoxyuridine 5′-triphosphate).


SEQUENCE LISTING (21 sequences)

SEQ ID NO: 1 - protein, 801 aa, Nanoarchaeum equitans
MLHQLPTMVVEEKAVKEEEGYSVLKCYWINIENTPLDEVILIGKDENNRACEVIIPYKWYFYFEGDIKDLEEFANNKKIKIEYTKEQKKYIEKPKDVYKVYVLHKHYPILKEFIKEKGYKKYETDINVYRKFLIDKGIEPFEWFEVEGKILLSTSNKVRIKAQSIKRLYEKTKPSVLAFDIEVYSEAFPNPEKDKIISIALYGDNYEGVISYKGEPTIKVNTEYELIEKFVEIIESLKPDIIVTYNGDNFDIDFLVKRASLYNIRLPIKLVNKKEPTYNFRESAHVDLYKTITTIYKTQLSTQTYSLNEVAKEILGEEKIYDYENMLYDWAIGNYNKVFEYNLKDAELTYKLFKYYENDLLELARLVNQPLFDVSRFSYSNIVEWYLIKKSRKYNEIVPNKPKMEEVERRKLNTYAGAFVYEPKPGLYENLAVLDFASLYPSIILEHNVSPGTIYCEHDDCKQNGVEAIINNEKKYVWFCKKVKGFIPTVLEHLYTKRLELKRKLKELDRDSEEYKIINAKQAVLKIIINATYGYMGFPNARWYCIDCAAAVAAWGRKYINYILKRAEEEGFKVIYGDTDSLFISGDKDKVLEFLEKVNKELPGKIQLDLEDFYVRGIFVKKRGEQKGAKKKYALLSEQGYIKLRGFEAVRTDWAPIVKEVQTKLLEILLKEGNIEKARQYIKEIIRKLRNREIPWEKLLITETIRKPLEKYKVEAPHVAAAKKYKRLGYKVMPGFRVRYLVVGSTGRVSDRIKIDKEVRGNEYDPEYYIEKQLLPAVEQILESVGIKDTFTGKKLTDFFK

SEQ ID NO: 2 - protein, 801 aa, Nanoarchaeum equitans
MLHQLPTMVVEEKAVKEEEGYSVLKCYWINIENTPLDEVILIGKDENNRACEVIIPYKWYFYFEGDIKDLEEFANNKKIKIEYTKEQKKYIEKPKDVYKVYVLHKHYPILKEFIKEKGYKKYETDINVYRKFLIDKGIEPFEWFEVEGKILLSTSNKVRIKAQSIKRLYEKTKPSVLAFDIEVYSEAFPNPEKDKIISIALYGDNYEGVISYKGEPTIKVNTEYELIEKFVEIIESLKPDIIVTYNGDNFDIDFLVKRASLYNIRLPIKLVNKKEPTYNFRESAHVDLYKTITTIYKTQLSTQTYSLNEVAKEILGEEKIYDYENMLYDWAIGNYNKVFEYNLKDAELTYKLFKYYENDLLELARLVNQPLFDVSRFSYSNIVEWYLIKKSRKYNEIVPNKPKMEEVERRKLNTYAGAFVYEPKPGLYENLAVLDFASLYPSIILEHNVSPGTIYCEHDDCKQNGVEAIINNEKKYVWFCKKVKGFIPTVLEHLYTKRLELKRKLKELDRDSEEYKIINAKQRVLKIIINATYGYMGFPNARWYCIDCAAAVAAWGRKYINYILKRAEEEGFKVIYGDTDSLFISGDKDKVLEFLEKVNKELPGKIQLDLEDFYVRGIFVKKRGEQKGAKKKYALLSEQGYIKLRGFEAVRTDWAPIVKEVQTKLLEILLKEGNIEKARQYIKEIIRKLRNREIPWEKLLITETIRKPLEKYKVEAPHVAAAKKYKRLGYKVMPGFRVRYLVVGSTGRVSDRIKIDKEVRGNEYDPEYYIEKQLLPAVEQILESVGIKDTFTGKKLTDFFK

SEQ ID NO: 3 - DNA, 2406 bp, Nanoarchaeum equitans
atgttacaccaactccccacgatggttgtagaagaaaaggcggtaaaagaggaagaagggtatagcgtgctaaaatgttattggattaatatagagaacacccctttagacgaggtaattttaataggtaaagacgaaaataatagagcttgtgaagttataattccatacaaatggtatttctattttgaaggcgatataaaggatttagaagaattcgctaacaacaaaaaaataaaaatcgaatatacaaaggagcaaaagaaatatatagaaaaaccaaaagatgtttataaagtatatgttttgcataaacattatccaatactaaaagaattcattaaagaaaagggctataaaaaatacgaaaccgatataaatgtttataggaagtttttaatagataaagggatagagccttttgaatggtttgaggtagaaggcaaaattttattatctacctctaacaaagttagaataaaagcacaaagtataaaaagattgtatgaaaagactaagccatcggttttagcttttgatatagaagtttacagtgaggctttccctaatcctgaaaaagacaaaataatatctatagccctttatggagacaattacgaaggggttatctcttacaaaggagaaccaactataaaagttaataccgaatatgaattaattgagaaatttgtcgaaataatagaaagcttaaaaccagacataatagttacatacaatggggataatttcgatatagactttttagtgaaaagggcttctttatacaatataaggctaccaataaaattggttaacaaaaaagagcctacttataattttagggaaagcgcacatgtagatttgtataaaacaattactaccatatataaaacccaattgtctacccaaacatattcattaaatgaagtagctaaagaaattcttggagaggagaaaatttatgattatgaaaacatgttatatgattgggccataggcaattataacaaagtgttcgaatacaatttaaaagatgccgaattaacatataagctattcaaatactatgaaaatgatttattggaattagcaagattggttaaccaaccattatttgatgtatctaggtttagctatagtaatatagttgaatggtatctaatcaaaaaaagcagaaaatataatgaaattgtgcctaacaaaccaaaaatggaagaagtagagagaagaaaattaaatacctatgcaggagcattcgtttacgaaccaaaacccggtttgtatgagaatttagctgtactggatttcgcttctctgtatccttcaattatattagagcataatgtttctccaggcacaatatattgtgagcatgatgattgtaaacaaaatggggtagaagcgataataaataatgagaaaaaatatgtgtggttttgcaaaaaagtaaaagggtttattccaacggtattagagcatttgtatacaaaaaggctagaacttaagagaaaactgaaagaactagatagggatagtgaagaatataaaattataaatgctaagcaaagagtattgaaaataataattaatgcaacctatggctatatgggtttcccaaatgcgagatggtattgcatagactgtgctgcggcagtagcagcttggggcaggaaatacattaattatatattaaaaagggccgaagaagaaggattcaaagtaatttatggagataccgattcattattcatttctggggacaaagacaaagtattagaatttttagagaaagtaaataaagaattacccggtaaaatacaattagatttagaagatttctatgttagagggatattcgtaaaaaagaggggtgaacaaaagggggcaaaaaagaaatatgctttattaagcgaacaaggttacataaagctaaggggcttcgaagcagtaagaacagactgggctcccatagttaaagaagtccaaacaaagctattggaaattttgctaaaagaaggtaacatagaaaaagcaagacaatacataaaagaaattattagaaagctaagaaatagagaaataccatgggagaagcttttaattacagaaacgataagaaagcctttagaaaaatacaaagttgaagctcctcatgtggcagcagcaaaaaaatataaaaggttgggctataaagttatgcctggctttagagttagatatttagtggtaggtagcactggaagggtttcagatagaattaaaatagacaaagaagttaggggtaatgaatatgaccccgaatactacatagaaaaacaactattgcctgcagtagagcaaatattagaatctgtaggtattaaagacacattcacaggcaaaaaactaacagatttctttaaatga

SEQ ID NO: 4 - DNA, 39 bp, Artificial Sequence
  Forward primer of A523R
tgctaagcaaagagtattgaaaataataattaatgcaac

SEQ ID NO: 5 - DNA, 40 bp, Artificial Sequence
  Reverse primer of A523R
ttttcaatactctttgcttagcatttataattttatattc

SEQ ID NO: 6 - DNA, 20 bp, Artificial Sequence
  Forward primer of Lambda-1
aataacgtcggcaactttgg

SEQ ID NO: 7 - DNA, 20 bp, Artificial Sequence
  Reverse primer of Lambda-1
gttacgccaccagtcatcct

SEQ ID NO: 8 - DNA, 20 bp, Artificial Sequence
  Forward primer of Lambda-2
caaaggcggttaaggtggta

SEQ ID NO: 9 - DNA, 20 bp, Artificial Sequence
  Reverse primer of Lambda-2
ggctgtaccggacaatgagt

SEQ ID NO: 10 - DNA, 20 bp, Artificial Sequence
  Forward primer of Lambda-3
agaagttcaggaagcggtga

SEQ ID NO: 11 - DNA, 20 bp, Artificial Sequence
  Reverse primer of Lambda-3
acggaaaaagagacgcagaa

SEQ ID NO: 12 - DNA, 20 bp, Artificial Sequence
  Forward primer of Lambda-4
ccggtaatggtgagtttgct

SEQ ID NO: 13 - DNA, 20 bp, Artificial Sequence
  Reverse primer of Lambda-4
gtactggtgcctttgccatt

SEQ ID NO: 14 - DNA, 20 bp, Artificial Sequence
  Forward primer of Lambda-5
tttacagcgtgatggagcag

SEQ ID NO: 15 - DNA, 20 bp, Artificial Sequence
  Reverse primer of Lambda-5
gacacggtggcttttgtttt

SEQ ID NO: 16 - DNA, 31 bp, Artificial Sequence
  NeqFP Primer
attatagcatatgttacaccaactccccacg

SEQ ID NO: 17 - DNA, 38 bp, Artificial Sequence
  NeqRP Primer
aaaaaactaacagatttctttaaatgagtcgacattat

SEQ ID NO: 18 - DNA, 24 bp, Artificial Sequence
  Hex-M13 Primer
ccgttgctaccctcgttccgatgc

SEQ ID NO: 19 - DNA, 24 bp, Artificial Sequence
  M13 Primer
gcatcggaacgagggtagcaacgg

SEQ ID NO: 20 - DNA, 20 bp, Artificial Sequence
  Forward primer of Beta-actin
agagatggccacggctgctt

SEQ ID NO: 21 - DNA, 20 bp, Artificial Sequence
  Reverse primer of Beta-actin
atgatggagttgaaggtagt